(19) United States
(12) Patent Application Publication
(10) Pub. No.: US 2002/0077354 A1
(43) Pub. Date: Jun. 20, 2002

(54) Methods and compositions for stabilizing oxidizable compounds

(76) Inventor: Dusan Miljkovic, San Diego, CA (US)

Correspondence Address: RUTAN & TUCKER, LLP, P.O. BOX 1950, COSTA MESA, CA, 92628-1950, US

(21) Appl. No.: 09/972,181

(22) Filed: Oct. 5, 2001

Related U.S. Application Data
(60) Provisional application No. 60/239,056, filed on Oct. 5, 2000.

(51) Int. Cl. A61K 31/375

(52) U.S. Cl. 514/474

(57) ABSTRACT

Methods and compositions employ an oxidizable compound with an electron donating group, and an electrophilic compound that accepts an electron from the electron donating group thereby forming a complex. The free oxidizable compound has a first stability towards oxidation and a second stability towards oxidation when bound in the complex, wherein the second stability is greater than the first stability.

This application claims the benefit of U.S. provisional application No. 60/239056, filed Oct. 5, 2000, which is incorporated herein by reference in its entirety

### FIELD OF THE INVENTION

Field of the invention is stabilization of oxidizable compounds.

### BACKGROUND

L-Ascorbic acid (Vitamin C) is one of the best-studied and most commonly consumed vitamins due to its various therapeutic and preventive qualities. Among other beneficial uses, Vitamin C is known to help prevent the common cold, to stimulate the synthesis of collagen, and to protect tissues against damaging external influences such as ultraviolet radiation and air pollution. Ascorbic acid has further been shown to exhibit potent antioxidative properties, and is known to react at relatively high concentrations with superoxide and hydroxyl radicals, which have been implicated as causative agents in various degenerative physiological processes such as destruction of lipid membranes, breakdown of DNA, and inactivation of enzymes.

Unfortunately, ascorbic acid has a pronounced tendency for oxidative degradation, especially in aqueous environments and various formulations and methods are known in the art to chemically or physically stabilize ascorbic acid. In one method, water is prevented from reacting with ascorbic acid. For example, Cantin et al. describe in U.S. Pat. No. 5,736,567 a cream preparation of ascorbic acid in which the water activity of the cream is below 0.85, and preferably below 0.7. In another example, U.S. Pat. No. 5,843,411 to Hernandez et al. water is entirely excluded from a vitamin C preparation. While exclusion of water may help prevent oxidative degradation at least to some extent, various applications or formulations may require an aqueous base, thereby significantly limiting the utility of water exclusion.

In another approach, air is excluded from vitamin C preparations by sealing single or multiple dosages in an airtight container (e.g., U.S. Pat. No. 5,846,996 to Fallick or U.S. Pat. No. 5,976,555 to Liu et al). Excluding air is a relatively effective method of preventing oxidation, however, typically has several disadvantages. For example, once a container is opened, the remaining quantity is no more protected. Furthermore, where the ascorbic acid is a part of a cream or other mixed preparation, the user has to admix a base with the ascorbic acid at the point of use.

In a still further approach, ascorbic acid is chemically derivatized to yield more stable compounds as taught in Japanese patent application 62/207289, WO 99/56720, published Nov. 11, 1999 (The Procter & Gamble Company), WO 00/28960, published May 25, 2000 (The Procter & Gamble Company), and WO 00/28961, published May 25, 2000 (The Procter & Gamble Company). However, chemical modification is relatively expensive, and often requires additional purification steps. Furthermore, not all chemical derivatives may be suitable for oral or systemic administration.

To avoid at least some of the problems associated with oral or systemic administration, non-toxic antioxidative additives may be included in vitamin C preparations as described in e.g., WO 97/10820, published Mar. 27, 1997 (The Green Cross Corporation), WO 98/00102, published Jan. 8, 1998 (Unilever N.V.), WO 99/07362, published Feb. 18, 1999 (Indústria Comércio de Cosméticos Natural LTD.), and WO 00/28960, published May 25, 2000 (The Procter & Gamble Company). Although additives frequently improve the shelf life of vitamin C preparations, additives tend to increase production cost, and are not equally well tolerated by all consumers.

Although there are many known methods to protect ascorbic acid from oxidative degradation, all or all of them have one or more disadvantages. Therefore, there is still a need to provide methods and compositions for stabilizing oxidizable compounds.

### SUMMARY OF THE INVENTION

A composition comprises an oxidizable compound with a first stability towards an oxidation, wherein the oxidizable compound has an electron donating group. The composition further includes an electrophilic compound that accepts an electron from the electron donating group, thereby forming a complex between the oxidizable compound and the electrophilic compound, wherein the oxidizable compound in the complex has a second stability towards the oxidation, and wherein the second stability is greater than the first stability.

In one aspect of the inventive subject matter, the oxidizable compound has a second electron donating group, and it is contemplated that the electron donating group and the second electron donating group are in a geminal, vicinal, or proximal position relative to each other. In further preferred oxidizable compounds, the oxidizable compound comprises an aromatic portion, and it is especially preferred that the oxidizable compound is ascorbic acid, salicylic acid, or a catechol derivative.

In another aspect of the inventive subject matter, the electron donating group comprises a hydroxyl group, a sulfhydryl group, a selenyl group, or an amino group, and it is preferred that the electrophilic compound comprises a metal or metal ion, more preferably from second, thirteenth, or fourteenth group of the periodic system, and even more preferably boron. Contemplated complexes also include electrically charged complexes, and particularly contemplated complexes are anionic complexes, which may or may not comprise a counter ion.

While it is generally contemplated that the second stability is greater than the first stability, it is especially preferred that the second stability is at least five times, fifty times, or five hundred times greater than the first stability. Particularly contemplated oxidation reactions comprise oxidations in aqueous systems.

### BRIEF DESCRIPTION OF THE DRAWING

FIG. 1 depicts various HPLC elution profiles of contemplated compounds and complexes.

### DETAILED DESCRIPTION

Compositions according to the inventive subject matter generally comprise an oxidizable compound with a first stability towards an oxidation, wherein the oxidizable compound further has an electron-donating group. Contemplated compositions further comprise an electrophilic compound that accepts one or more electrons from the electron-donating group, thereby forming a complex between the oxidizable compound and the electrophilic compound, wherein the oxidizable compound in the complex has a second stability towards the oxidation, and wherein the second stability is greater than the first stability. As used herein, the term “accepts electron” refers to formation of a donating bond, that is, a non-covalent and non-ionic bond between an electron-rich compound and an electron-poor and/or electron accepting (e.g., in an unoccupied orbital) compound. Consequently, the term “electrophilic compound” includes positively charged compounds, and neutral or negatively charged compounds that can act as electron acceptor in a complex, non-ionic or other bond.

With respect to the oxidizable compound, it is generally contemplated that all oxidizable compounds are suitable for use in conjunction with the teachings presented herein, however, particularly contemplated oxidizable compounds have a second electron donating group which may be in a geminal, vicinal or proximal position to relative to each other. The term “proximal” as used herein refers to any position of two groups that allows both of the groups to simultaneously form a complex with an electrophilic compound. For example, functional groups in a ribofaranose at carbon atoms 1 and 3, or 1 and 4 are considered to be in a proximal position. In contrast, two groups spaced apart by an inflexible molecular scaffold that keeps the groups at least 50Å apart is not considered proximal under the scope of this definition.

It is generally contemplated that the oxidizable compounds may further comprise one or more unsaturated portions, which may or may not be conjugated, or which may or may not form an aromatic system. Particularly preferred oxidizable compounds include pharmacologically, nutritionally and cosmetically relevant molecules. For example, pharmacologically relevant molecules include salicylic acid and derivatives, but also substituted and unsubstituted flavonoids, catechins, and terpenoids. Nutritionally relevant molecules include ascorbic acid and derivatives, and cosmetically relevant molecules include substituted and unsubstituted ascorbic acid and (poly)glycolic acids. Typical representatives of preferred compounds may further include substituted and unsubstituted quercetin, rutin, 3-hydroxyflavones, kojic acids, kaempferol, etc. Still further contemplated oxidizable compounds include compounds of veterinarian and/or agricultural use that are prone to complete or partial oxidative degradation. Moreover, it should also be appreciated that all of the contemplated oxidizable compounds may be in a pro-form (e.g., pro-drug that is hydrolyzed or otherwise activated), in oligomeric or polymeric form, and/or in covalent or non- covalent association with a non-oxidizable compound.

With respect to the electron donating group it is contemplated that all groups or moieties that can form a complex bond with an electrophilic compound are appropriate, and especially contemplated electron donating groups include hydroxyl groups, sulfhydryl groups, selenyl groups, and amino groups. Typically, contemplated oxidizable compounds include at least one or two electron donating groups, and in some preferred oxidizable compounds the electron donating groups are in a vicinal position and/or conjugated with at least one double bond. Exemplary preferred configurations of electron donating groups are depicted below (Structures 1-3), in which R′ and R″ are independently selected from substituted or unsubstituted alkyl, alkenyl, alkynyl, aryl, alkaryl, all of which may or may not comprise a heteroatom (e.g, O, S, N, Se, P), and wherein R′ and R″ may together form a substituted or unsubstituted ring:

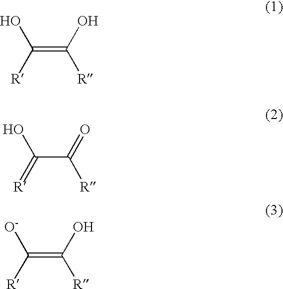

Consequently, particularly preferred electron donating groups are moieties within a substituted or unsubstituted carbocyclic ring system, which may additionally include at least one double bond, and which may further be an aromatic carbocyclic ring system. Such contemplated ring systems may further include a heteroatom (typically O, but S, Se, P, and N are also contemplated). Moreover, where stereochemical orientation of two electron donating groups can be differentiated (e.g., in alpha or beta orientation relative to a plane drawn through the molecule), all chemically reasonable combinations of stereochemical orientations are contemplated.

In a further contemplated aspect of the inventive subject matter, the electrophilic compound preferably comprises an (electrophilic) element, and more preferably a group 13 element such as boron. While not limiting to boron as the contemplated element, it should be appreciated that suitable elements may be bound in a covalent and/or complex bond in the electrophilic compound. Consequently, contemplated electrophilic compounds may derive their electrophilicity from the element, the element bound to the compound, and/or from one or more groups in the compound independent of an element (which may or may not be present in the compound).

It should further be recognized that contemplated elements (and particularly boron) may be bound to the electrophilic compound via one or more monodentate, bidentate, tridentate, or tetradentate ligands. In an especially contemplated aspect of the inventive subject matter, preferred electrophilic compounds include those in which the element, and particularly boron, is bound to a carbohydrate (e.g., various pentoses or hexoses in furanose or pyranose form) or an amino acid (e.g., serine, threonine, etc.), and exemplary complexes including ascorbate and boron-containing compounds are depicted below in Structures 4 and 5. However, it should also be recognized that two or more oxidizable compounds may form a complex with borate (see below). Further particularly contemplated complexes including boron are described in U.S. Pat. Nos. 5,962,049, 5,985,842, and 6,080,425 to Miljkovic, all of which are incorporated by reference herein.

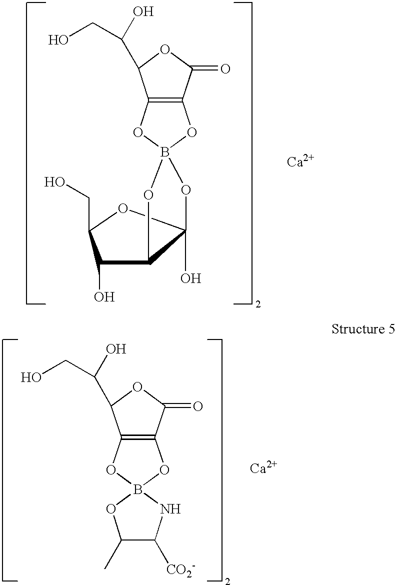

However, alternative electrophilic compounds need not necessarily be limited to those comprising a metal, and various alternative electrophilic compounds include organic, organometallic and inorganic molecules. For example, organic electrophiles may include quaternary ammonium compounds, sulfonium compounds, etc., while organometallic molecules may include organosilicon compounds, and inorganic compounds may include silica.

With respect to the complex between the oxidizable compound and the electrophilic compound, it is generally contemplated that the complex is formed between at least one electron donating group and an electrophilic compound. However, it should be recognized that alternative complexes may also be formed from all reasonable stoichiometric ratios between electron donating groups and electrophilic compounds. For example, suitable complexes may be formed between three or more electron donating groups and single electrophilic compound. On the other had, appropriate complexes may also be formed from two or more electrophilic compounds and one or more electron-donating group. It is furthermore contemplated that suitable complexes have a K_{A} (association constant defined as [Complex_{Oxidizable Compound/Electrophilic compound}]/[Electrophilic compound]×[Oxidizable Compound] in M⁻¹) of at least 100, more preferably 500, even more preferably more than 1000, and most preferably more than 5000.

It should also be appreciated that suitable complexes may have an electric charge, and it is particularly contemplated that the electric charge is a negative charge. Consequently, it is contemplated that the complex charge may be compensated by a counter ion, and where the complex charge is negative, especially contemplated counter ions include potassium, sodium, ammonium, calcium, and trimethyl-methyl-ammonium.

With respect to the stability against oxidation, it is generally contemplated that the oxidizable compound has a first stability towards an oxidation, and that the oxidizable compound, when in the complex, has a second stability towards an oxidation, wherein the second stability is greater than the first stability. As used herein, the term “first stability” refers to the intrinsic stability of the compound in a particular environment (e.g., dissolved in water at pH7 and 20° C.) for a predetermined period (e.g., 96 hours), while the term “second stability” refers to the joint stability of the compound in complex with the electrophilic compound in the same environment for the same predetermined period. As further used herein, the term “stability towards an oxidation” refers to a chemical resilience of a compound to partial or complete degradation that involves (a) a reaction with oxygen at least in some form, or (b) a reaction in which the compound looses one or more electrons. There are many chemically distinct degradation reactions possible for the entire gamut of contemplated oxidizable substances. For example, a typical oxidation reaction includes a non-enzymatic reaction of an alcohol group into a keto group in an aqueous system. Therefore, chemical resilience against degradation is advantageously measured in the amount of remaining, undegraded oxidizable compound over a predetermined period.

It is especially contemplated that the second stability is at least five times (i. e., at least five times more undegraded oxidizable compound in the complex as compared to oxidizable compound alone over a predetermined period), preferably at least fifty times (i.e., at least fifty times more undegraded oxidizable compound in the complex as compared to oxidizable compound alone over a predetermined period), and more preferably at least five hundred times (i.e., at least five hundred times more undegraded oxidizable compound in the complex as compared to oxidizable compound alone over a predetermined period) greater than the first stability. While not wishing to be bound to a particular theory or hypothesis, it is contemplated that the increased stability of the oxidizable compound towards oxidation is at least in part due to the decreased availability of electrons in the oxidizable compound to an oxidative reagent.

Consequently, a method of increasing chemical stability of a compound has a first step in which an oxidizable compound is provided having a first stability towards an oxidation, and wherein the oxidizable compound has an electron-donating group. In a further step, an electrophilic compound is provided that accepts an electron from the electron donating group, and in a subsequent step a complex is formed between the oxidizable compound and the electrophilic compound, wherein the oxidizable compound in the complex has a second stability towards the oxidation, and wherein the second stability is greater than the first stability.

With respect to the step of providing the oxidizable compound and the electrophilic compound, it is generally contemplated that suitable compounds may be purchased in bulk or minor amounts, or synthesized in a laboratory, and the particular method of providing typically depends on the particular compound chosen.

It is generally preferred that the complex is formed in a single step reaction that involves suitable amounts of at least both electrophilic compound and oxidizable compound, and it is further contemplated that the reaction mixture may further comprise additional reactive or non-reactive components, including catalysts (e.g., acid, base), solvents (preferably water), fillers, coloring agents, indicators, salts for counter ions, etc. Typically, the formation will include a step of activation, such as heating, cooling, or pressurization. However, in alternative aspects of the inventive subject matter, the formation of the complex may vary substantially, and the particular reaction conditions will depend on the particular reactants. For example, the complex formation may involve a multi-step reaction that may or may not include intermediate purification steps.

In particularly preferred aspects of the inventive subject matter, contemplated compounds and methods may advantageously be employed to impart stability against oxidation into readily oxidizable compounds and compositions including such compounds. For example, where the oxidizable compound is a nutritional supplement (e.g., ascorbic acid, or a flavonoid), it is contemplated that the shelf life of such compounds may be significantly increased by forming contemplated complexes, wherein the complexes may either be in isolated form (e.g., Vitamin C tablet) or be a component in a nutritional item (e.g., multivitamin drink comprising Vitamin C). Consequently, contemplated methods also include methods of stabilizing an oxidizable component in a food item or nutritional supplement by forming a complex of the oxidizable component with an electrophilic compound. Thus, it is contemplated that food items and nutritional supplement may include contemplated compounds and complexes.

Similarly, it should be appreciated that stabilization of an oxidizable compound need not be limited to a nutritional supplement, and it is particularly contemplated that oxidizable compounds also include compounds for cosmetic use, and particularly ascorbic acid. Consequently, contemplated methods also include methods of stabilizing an oxidizable component in a cosmetic product or cosmetic formulation by forming a complex of the oxidizable component with an electrophilic compound. Thus, it is contemplated that cosmetic products and formulations (e.g., skin 20 creams) may include contemplated compounds and complexes.

Experiments

The following experiments illustrate in an exemplary manner methods and compositions of the inventive subject matter.

### Potassium Boro-Ascorbate

L-Ascorbic Acid (256 gr; 2 moles) is dissolved in 800 ml of distilled water. Boric Acid (62 gr 1 mole) is added to this solution and with stirring potassium bicarbonate (200 gr; 2 moles) was slowly and gradually added to the reaction mixture over 30 minutes. Vigorous evolution of carbon dioxide occurs. The reaction mixture is stirred at room temperature for another hour and then the clear colorless solution is freeze-dried (lyophilized).

Potassium Boro-Ascorbate remains as a white crystalline solid (350 gr; practically quantitative yield). The purity of the product is very high (according to HPLC; C₁₈ reverse phase column; phosphate buffer; pH=6.3; UV-detection). If necessary, the compound can be re-crystallized from ethanol.

### Sodium Boro-Ascorbate

This compound has been essentially made in the same manner as the potassium salt (instead of 200 gr of potassium bicarbonate, 168 gr of sodium bicarbonate (2 moles) were used, with a final yield of 315 gr).

### Calcium Boro-Ascorbate

This compound was made in analogous way as the two previous examples. As a base one uses Ca-carbonate (one mole per two moles of ascorbic acid and one mole of boric acid). The yield of the final Ca-boro-ascorbate was 310 gr.

### Stability of Boro-Ascorbate

Sodium boro-ascorbate was prepared as described above and 100 ml of a 10 mM aqueous solution was prepared by dissolving appropriate quantities of sodium boro ascorbate in deionized water. The pH was adjusted to 7.0 and the so prepared solution was divided into two portions. The first portion was kept at room temperature (about 21° C.), while the second portion was incubated in a temperature controlled water bath at 45° C. Both portions were kept at their respective temperatures for 7 days, and subsequently analyzed via HPLC. For control, a 10 mM aqueous ascorbate solution was prepared as described above and incubated at room temperature. In a further experiment, a skin cream formulation (Glycerol mono-oleate (readily commercially available) (5 g), polyglycerol oleate (“Plurol Oleic”, “Gatte Fosse Inc”) (5 g), soybean lecithin granules (0.5 g), sodium cholate (0.3 g), tocoferol (0.5 g), gelucir 44 (2 g) and almond oil (10 g) are fully homogenized in a mixer at 40-rpm (component A). Separately, ascorbic acid (0.5 g), sodium bicarbonate (0.25 g) and boric acid (0.1 g) are stirred in water (20 ml) for 0.5 hour at room temperature providing a clear solution (component B). Component B is added gradually to component A while slowly mixing in a mixer at 40 rpm during 1 hour at room temperature. The product is a cream formulation containing 2,77 mg B per gram.) was prepared, and incubated at room temperature for seven days.

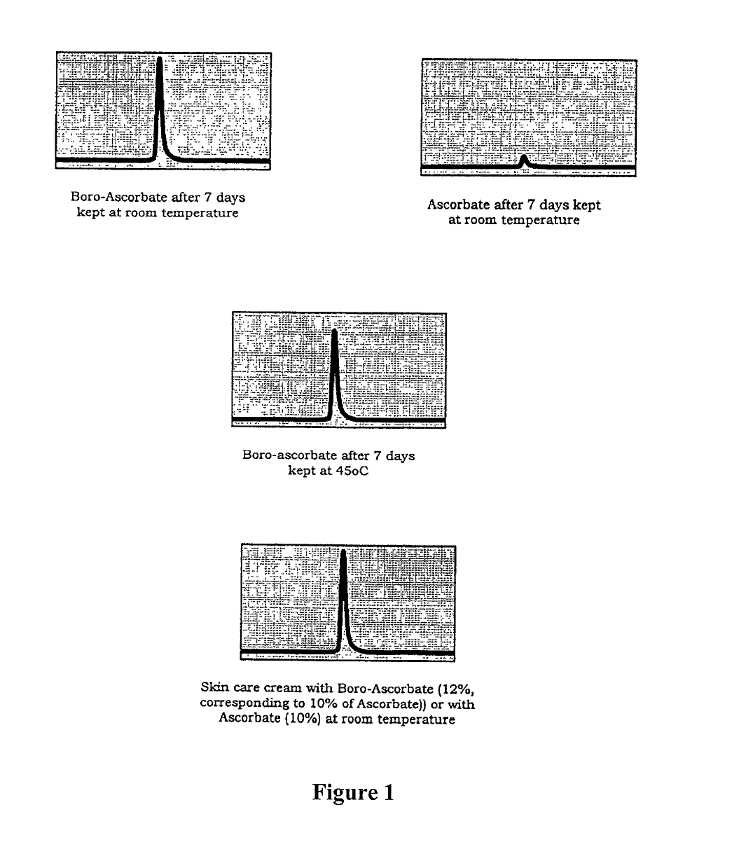

All incubations were then subjected to HPLC analysis. FIG. 1 depicts the respective elution profiles, which clearly indicate stabilization of ascorbic acid in contemplated compositions (i.e., that the oxidizable compound in complex with the electrophilic compound has a higher stability towards oxidation than the oxidizable compound by itself).

### Further Contemplated Exemplary Compounds

Potassium Boro-Salicylate and Potassium di-catecholo-borate have been prepared in analogous fashion. It should further be contemplated that a fraction of the ascorbic acid (preferably 50 mol % of the ascorbic acid) may be replace with other compounds that may form a complex with boron such that at least a fraction of the reaction products will include a mixed complex between ascorbate, boron, and the other compound. For example, it is contemplated that suitable mixed complexes may be formed with various carbohydrates and/or amino acids as depicted in Structures 4 and 5 above.

Thus, specific embodiments and applications of methods and compositions for stabilizing oxidizable compounds have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A composition, comprising:
an oxidizable compound having a first stability towards an oxidation, the oxidizable compound further having an electron donating group;
an electrophilic compound that accepts electrons from the electron donating group, thereby forming a complex between the oxidizable compound and the electrophilic compound, wherein the oxidizable compound in the complex has a second stability towards the oxidation; and
wherein the second stability is greater than the first stability.

2. The composition of claim 1 wherein the oxidizable compound includes a structure according to structures 1-3 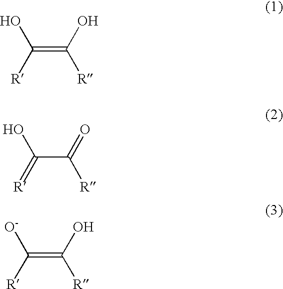
wherein R′ and R″ are independently selected from a substituted or unsubstituted alkyl, alkenyl, alkynyl, aryl, alkaryl;
wherein R′ and R″ optionally comprise a heteroatom selected from the group consisting of O, S, N, Se, and P; and
wherein R′ and R″ optionally together form a substituted or unsubstituted ring.

3. The composition of claim 1 wherein the oxidizable compound further comprises a second electron donating group in a position relative to the first electron donating group selected from the group consisting of a geminal position, a vicinal position, and a proximal position

4. The composition of claim 3 wherein the electron donating group and the second electron donating group are in a vicinal position relative to each other.

5. The composition of claim 3 wherein the electron donating group in the oxidizable compound is in conjugation with at least one double bond in the oxidizable compound.

6. The composition of claim 3 wherein the oxidizable compound is selected from the group consisting of an ascorbic acid, a salicylic acid, and a catechin.

7. The composition of claim 1 wherein the electron-donating group comprises a group selected from the group consisting of a hydroxyl group, a sulfhydryl group, a selenyl group, and an amino group.

8. The composition of claim 1 wherein the electrophilic compound comprises a metal.

9. The composition of claim 8 wherein the metal is group thirteen metal.

10. The composition of claim 8 wherein the electrophilic compound is a borate or a silicate.

11. The composition of claim 1 wherein the second stability is at least five times greater than the first stability.

12. The composition of claim 1 wherein the second stability is at least fifty times greater than the first stability.

13. The composition of claim 1 wherein the second stability is at least five hundred times greater than the first stability.

14. The composition of claim 1 wherein the oxidation comprises a reaction of an alcohol group in the oxidizable compound into a keto group in an aqueous system.

15. The composition of claim 1 wherein the complex is an anionic complex.

16. The composition of claim 15 further comprising a counter ion selected from the group consisting of a potassium cation, a sodium cation, an ammonium cation, a calcium cation, and a trimethyl-methyl-ammonium cation.

17. A method of increasing chemical stability of a compound, comprising:
providing an oxidizable compound having a first stability towards an oxidation, the oxidizable compound further having an electron donating group;
providing an electrophilic compound that accepts an electron from the electron donating group;
forming a complex between the oxidizable compound and the electrophilic compound, wherein the oxidizable compound in the complex has a second stability towards the oxidation, and wherein the second stability is greater than the first stability.

18. The method of claim 17 wherein the oxidizable compound has a second electron donating group, and wherein the electron donating group and the second electron donating group are in a position relative to each other selected from the group consisting of a geminal position, a vicinal position, and a proximal position.

19. The method of claim 17 wherein the electrophilic compound comprises a metal atom.

20. The method of claim 17 wherein the oxidizable compound is selected from the group consisting of an ascorbic acid, a salicylic acid, and a catechin, and wherein the electrophilic compound comprises a borate, and wherein the second stability is five hundred times greater than the first stability.
